(19) United States
(12) Patent Application Publication
(10) Pub. No.: US 2006/0234268 A1
(43) Pub. Date: Oct. 19, 2006

(54) LINEAR AMPLIFICATION OF RNA

(76) Inventors: Sungwhan An, Daejon (KR); Chiwang Yoon, Daejon (KR); Youngho Moon, Daejon (KR); Tae Jeong Oh, Daejon (KR); Dae Kyoung Yoon, Daejon (KR); Myungsoon Kim, Daejon (KR)

Correspondence Address: JHK LAW, P.O. BOX 1078, LA CANADA, CA, 91012-1078, US

(21) Appl. No.: 11/379,047

(22) Filed: Apr. 17, 2006

Related U.S. Application Data
(60) Provisional application No. 60/594,532, filed on Apr. 15, 2005.

(51) Int. Cl. C12Q 1/68; C12P 19/34

(52) U.S. Cl. 435/6; 435/91.2

(57) ABSTRACT

The present application discloses a method for linear amplification of RNA.

### CROSS-REFERENCE To RELATED APPLICATIONS

The present application claims the benefit of priority to U.S. Provisional Patent Application No. 60/594,532, filed Apr. 15, 2005. The present application also claims the benefit of priority to U.S. patent application Ser. No. 10/984,640, filed Nov. 9, 2004, the contents of which are incorporated by reference in their entirety.

### BACKGROUND OF THE INVENTION

1. Field of the Invention

The present invention relates to a method for the linear amplification of RNA using a high-heel primer, and more particularly to a method for the linear amplification of a small amount of RNA, in which annealing and extension are performed at the same temperature using a high-heel primer.

2. General Background and State of the Art

In the molecular diagnostic field, as the need increases to perform diagnosis on a small amount of sample, using non-invasively obtained samples such as stool, sputum and the like, rather than applying prior invasive methods, there is a correspondingly increasing need for technology to amplify a very small amount of sample.

Recently, there were reports that the amplification of RNA sample results in an increase in sensitivity and a decrease in technical variation, so as to improve the quality of test results, thereby facilitating more meaningful biological discovery (Park, P J et al., J. Biotechnol., 112:225, 2004; Feldman, A L et al., Biotechniques, 33:906, 2002; Polacek, D C et al., Physiol Genomics, 13:147, 2003; Kurn, N and Heath, J D, Genetic Engineering News, 24(3), 2004). As a result, a highly sensitive, standardized method for the amplification of RNA sample, which can be applied routinely regardless of sample, is necessary to perform successful microarray tests.

One of the most important considerations in a sample preparation process for diagnosis and the study of gene expression patterns is that the relative representation of all transcripts present in the sample must be efficiently performed regardless of a total RNA used. From this viewpoint, the linearity of an amplification method that is used is very important.

As an amplification method for preparing a microarray sample, an in vitro transcription method using a T7 polymerase-based primer is most generally used (van Gelder, R N et al., PNAS, 87:1663, 1990; Glanzer, J G and Eberwine, J H, Br. J. Cancer, 90:1111, 2004). Although this transcription method satisfies the amount required in a microarray test, it still has critical limitations that can deteriorate the quality of test data. First, since a test process is complex, time longer than 4 days may be required to prepare a microarray sample. Second, at least two amplification steps are required to obtain a sufficient amount of sample for microarray tests from less than 100 ng of total RNA. In such amplification steps, many technical variations can occur. Furthermore, samples subjected to amplification steps of different numbers in relation to one another will provide targets of correspondingly different amounts, so as to cause an error between a larger amount of sample and a smaller amount of sample, thus reducing the reliability of test data.

There have been various attempts to develop effective linear amplification methods, but such attempts have not departed greatly from the T7 polymerase-based amplification method as described above. Recently, a new RNA amplification method using an isothermal enzyme was developed by Nugen Co. (WO 02/072772 A2; U.S. Pat. No. 6,251,639B1). This is a method of performing linear amplification using DNA/RNA primers and is advantageous in that it can amplify even a small amount (about 1 ng) of sample and has high reproducibility. However, it has a disadvantage in that it is too expensive to use as a standardized target preparation method for gene expression analysis, which generally requires large amounts of tests.

### SUMMARY OF THE INVENTION

Accordingly, the present inventors have conducted extensive studies to develop a more effective method for the linear amplification of a small amount of RNA sample, and consequently found that, when a high-heel primer is used and primer-template binding (annealing) and cDNA extension are performed at the same temperature, specificity and sensitivity can be maximized and RNA sample can be linearly amplified within a short time at a low cost, thereby perfecting the present invention.

The present invention provides a linear amplification method that allows a sufficient amount of target for microarray tests to be obtained from a small amount of RNA sample.

The present invention in one aspect relates to a method for the linear amplification of RNA, including the steps of: (a) adding a poly dT-high heel primer, obtained by binding poly dT to the 3′-terminal end of a high-heel primer, to sample RNA, and then allowing the mixture to react at a temperature of 65-75° C. so as to anneal the poly dT-high heel primer with the poly-A portion of the sample RNA; (b) reacting the annealed sample with reverse transcriptase reactant so as to synthesize a cDNA, thus forming an RNA/cDNA hybrid; (c) reacting the formed RNA/cDNA hybrid with enzyme reactant including RNaseH, DNA polymerase and DNA ligase, so as to synthesize a double-stranded cDNA; and (d) adding a high-heel primer, dNTP and DNA polymerase to the double-stranded cDNA and subjecting the mixture to linear PCR amplification, in which annealing and extension are performed at the same temperature (65-75° C.).

In another aspect, the present invention relates to a method for the linear amplification of RNA, including the steps of: (a) adding a poly dT-high heel primer, obtained by binding poly dT to the 3′-terminal end of a high-heel primer, to sample RNA, and then allowing the mixture to react at a temperature of 65-75° C. so as to anneal the poly dT-high heel primer with the poly-A portion of the sample RNA; (b) reacting the annealed sample with reverse transcriptase reactant so as to synthesize a cDNA, thus forming an RNA/cDNA hybrid; (c) adding an enzyme for removing the RNA from the RNA/cDNA hybrid to the formed RNA/cDNA hybrid, so as to cut the RNA from the hybrid; (d) reacting the remaining cDNA with DNA polymerase, dNTP and DNA ligase, so as to synthesize a double-stranded cDNA; and (e) adding a high-heel primer and DNA polymerase to the synthesized double-stranded cDNA and subjecting the mixture to linear PCR amplification, in which annealing and extension are performed at the same temperature (65-75° C.).

In the preferred practice of the present invention, the high-heel primer is preferably represented by SEQ ID NO: 1, and the poly dT-high heel primer is preferably represented by SEQ ID NO: 2. The amount of the sample RNA is preferably nanogram level. The reverse transcriptase reactant preferably includes reverse transcriptase, dNTP mixture, and RNAsin which is an RNase inhibitor. The step of amplifying the double-stranded cDNA may additionally comprise adding aminoally1-dUTP to the cDNA and labeling the cDNA with a monofunctional fluorescent substance. In the step of forming the RNA/cDNA hybrid, a portion excepting the poly dT in the poly dT-high heel primer is preferably not hybridized with RNA.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 is a schematic diagram showing the inventive method for the linear amplification of RNA using a high-heel primer.

FIG. 2 shows the results of linear amplification for each 1 μg of total RNAs of a control group [293 (ATCC CRL1573)] and a test group [HeLa (ATCC CCL2)] according to the present invention. (A) shows the confirmation of final products by agarose gel electrophoresis, and (B) shows the results of 17K cDNA microarray test on final products which have been purified and labeled with a fluorescent dye.

FIG. 3 shows the confirmation of reproducibility of the inventive linear amplification method for a small amount (1 μg) of total RNA. (A) shows the results of 17K human cDNA microarray test on target which had been subjected to two independent linear amplification processes (1st & 2nd), and the results of the same microarray test only using a control group 293 (ATCC CRL1573) RNA (yellow test). The left graph of (B) numerically shows the results of (A), and shows the correlation between two amplification tests (1st and 2nd amplifications) on selected 6928 genes that have a signal intensity of more than a given value in the two tests, and the right graph of (B) shows the correlation of test results on 293 (ATCC CRL1573) RNA which have been coupled with Cy3 and Cy5 dyes.

FIG. 4 shows hybridization results for a control group [293 (ATCC CRL1573)] and a test group [HeLa (ATCC CCL2)]. The left image of (A) shows an array where 100 μg of total RNA have been hybridized with directly labeled cDNA, and the right image shows the same section of an array where 1 μg of total RNA have been subjected to linear amplification and then hybridized with cDNA. (B) is a graphic diagram which numerically shows the correlation between test results for 100 μg of total RNA and linear amplification test results for 1 μg of total RNA.

FIG. 5 shows microarray test results for each of amplification products which have been linearly amplified, for varying amounts (1 μg, 5 μg, 10 μg and 20 μg) of RNA.

FIG. 6 shows the sensitivity of the inventive linear amplification method. (A) shows microarray results for target obtained by subjecting nanogram level (200 ng and 20 ng) of RNA to linear amplification, and (B) is a graphic diagram that numerically shows the correlation between tests.

### DETAILED DESCRIPTION OF THE PREFERRED EMBODIMENTS

In the present application, “a” and “an” are used to refer to both single and a plurality of objects.

The present invention relates to a method for the linear amplification of RNA using a high-heel primer, which is entirely different from the prior T7 polymerase-based amplification method. The inventive method not only allows a sufficient amount of target for microarray tests to be obtained from a nanogram level of total RNA by one linear amplification process, but also has high sensitivity. Moreover, the inventive method is not only time-efficient, but also convenient enough to apply routinely to a process for microarray sample preparation.

As used herein, the term “high-heel primer” is defined as an oligonucleotide with a Tₘ of about 65 to about 75° C., preferably about 65 to 74, 65 to 73, 65 to 72, 65 to 71, 65 to 70, or preferably about 66 to 75, 66 to 74, 66 to 73, 66 to 72, 66 to 71, 66 to 70, or preferably 67 to about 75, 67 to 74, 67 to 73, 67 to 72, 67 to 71, 67 to 70, or preferably, about 68 to 75, 68 to 74, 68 to 73, 68 to 72, 68 to 71, 68 to 70, or preferably, 69 to 75, 69 to 74, 69 to 73, 69 to 72, 69 to 71, 69 to 70, or preferably 70 to 75, 70 to 74, 70 to 73, 70 to 72, 70 to 71, or preferably, 71 to 75, 71 to 74, 71 to 73, 71 to 72, or 72 to 75, 72 to 74, 72 to 73, or preferably about 72 degrees C. The high-heel primer may contain a GC content of more than about 70%, preferably, 72%, 74%, 76%, 78%, 80%, 82%, 84%, 86%, 88%, 90%, 92%, 94%, 96%, or 98%. The length of the high-heel primer oligonucleotide is not limited to any specific length or composition or temperature of melting so long as the oligonucleotide functions to anneal to the template nucleic acid and extend the bound primer at about the same temperature. Thus, the length and composition of the primer oligonucleotide may be manipulated and optimized according to the desire of the practitioner to achieve a particular primer annealing and extension reaction temperature. In one aspect of the invention, the “high-heel” portion of the primer may be from about 10 nucleotides to about 50 nucleotides long. Preferably, the length may be from about 10 to about 40, 10 to 35, 10 to 30, 10 to 25 or 10 to 20. The high-heel portion of the primer may be from about 15 to about 50, 15 to 40, 15 to 35, 15 to 30, 15 to 25, 15 to 20. The length of the high-heel primer may not be limited to any fixed length so long as the annealing of the primer oligonucleotide optionally linked to a poly-dT tail and its primer extension reaction are carried out at the same on about the same temperature. By “about” the same temperature, there is provided certain latitude where a one or two degree deviation is allowed, which may be caused by environmental conditions that are not strictly controlled.

A high heel primer-based linear amplification system according to the present invention is suitably prepared as follows. To synthesize a first strand cDNA, a poly dT-high heel primer, obtained by binding poly dT to the 3′-terminal end of a high-heel primer, is added to a sample RNA, and allowed to react at suitable elevated temperature, e.g., of 65-75° C., so as to anneal the poly dT-high heel primer with the poly-A portion of the sample RNA. The annealed sample is added with reverse transcriptase reactant and allowed to react, thus synthesizing a first strand cDNA.

Next, a reaction for synthesizing a second strand cDNA is performed. In this reaction, the first strand cDNA synthesized as described above is added with RNase H, E. coli DNA ligase, and DNA polymerase, so as to synthesize a double-stranded cDNA. In this regard, the RNase H specifically recognizes and cuts only RNA hybridized with DNA, and the resulting RNA fragments act as random primers, thus synthesizing the double-strand DNA.

The sense strand of the synthesized double-strand DNA is used as a template in a linear amplification step, in which this DNA contains the sequence of a high-heel primer at the 3′-terminal end. For this reason, the double-strand DNA is added with a high-heel primer, dNTPs (dATP, dCTP, dGTP, dTTP, and aminoally1-dUTP) and DNA polymerase, and subjected to linear PCR amplification, in which annealing and extension are performed at the same elevated temperature (65-75° C.) so that the linear amplification of RNA can be efficiently achieved.

The high-heel primer-based amplification system according to the present invention has the following conveniences.

First, unlike the prior T7 polymerase-based amplification method, all the steps of synthesizing a target for microarray tests are based on DNA level. Furthermore, this system produces a stable single-strand cDNA as a final product, unlike the prior system, which produces an unstable RNA product that has the possibility of degradation. RNA products always have a risk that can cause a difference in gene expression between samples, due to nonspecific damage to labeled RNA during a process for sample preparation or storage, whereas stable DNA targets can minimize this risk.

Second, the inventive high-heel primer-based amplification system not only provides the relative representation of a difference in the expression of transcripts between samples different from each other in the most exact manner, but also minimizes an error in gene expression according to total RNA conditions and RNA amounts that are different from each other between the same samples, thus maintaining the reliability and linearity of tests.

Third, the inventive system is cost-effective as compared to any prior system. Specifically, in accordance with the present invention, a routine sample preparation system for microarray tests can be provided at a low cost without constructing a special system.

Fourth, the inventive system is highly time-efficient. Due to the convenience of its total system, the inventive system allows a sufficient amount of target for microarray tests to be obtained from a nanogram level of sample by only one amplification step without requiring additional time, thus minimizing technical error rates in test procedures. As described above, target preparation by methods different from each other has a risk that can cause a statistical error in an analysis process for microarray tests. For this reason, not only a small amount of sample, but also a sufficient amount of sample, is preferably tested by the same method. Furthermore, there are significant differences in the activity and labeling efficiency of enzyme, and in sensitivity and the like, between sample preparation methods. As a result, based on the above-described considerations, a routine, standardized sample preparation method can be considered necessary to eliminate technical variations.

Hereinafter, the inventive method for the linear amplification of cDNA from a small amount of RNA sample will be illustratively described in further detail.

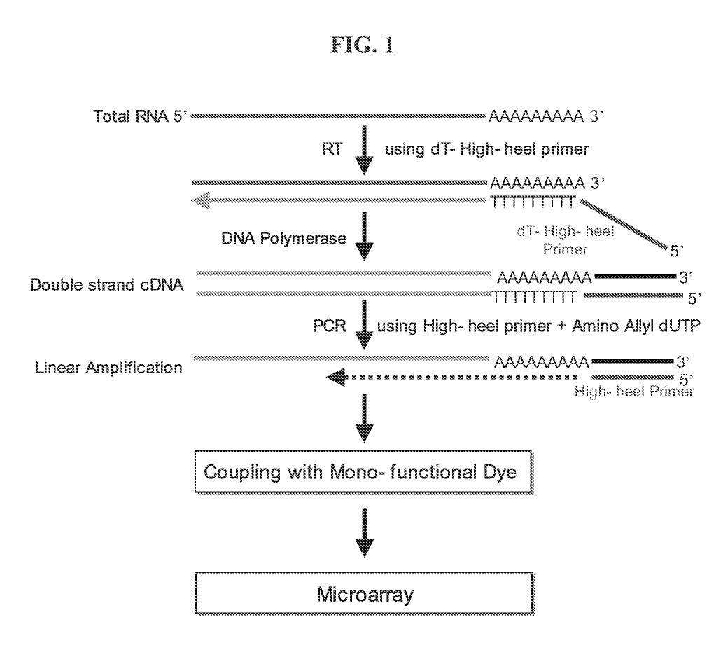

FIG. 1 is a schematic diagram illustrating the inventive method for the linear amplification of cDNA from a small amount of RNA sample. As shown in FIG. 1, a poly dT-high heel primer is annealed in the poly-A portion of sample mRNA, and then a single-strand cDNA is synthesized from the annealed mRNA using reverse transcriptase.

The poly dT-high heel primer is a primer obtained by binding a CTG base and poly dT to the 5′- and 3′-terminal ends of a high-heel primer used in linear amplification, respectively. The poly-dT portion has a base sequence of 3n+1 (n=5, 6, 7, 8, 9 or 10) bases, in which the (3n)th base is one base selected from the group consisting of A, G and C, and the (3n+1)th base is one base selected from the group consisting of A, T, G and C. The poly dT-high heel primer is annealed at a temperature, e.g., of 65-75° C. and most preferably 72° C. When an RNA/cDNA hybrid is formed, a portion excepting the poly dT in the poly dT-high heel primer is not hybridized with RNA.

The formed RNA/cDNA hybrid is added with RNase H, E. coli DNA ligase, and DNA polymerase, so as to synthesize a double-strand cDNA. At this time, the RNase H specifically recognizes and cuts only RNA hybridized with DNA, and the resulting RNA fragments act as random primers, thus synthesizing the double-strand DNA.

The obtained double-stranded cDNA is added with a high heel primer and DNA polymerase and then subjected to linear amplification, thus obtaining a large amount of amplification products from a small amount of sample.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Synthesis of cDNA by Reverse Transcription Reaction

2 μg of a poly dT-high heel primer of SEQ ID NO: 2, obtained by binding poly dT to the 3′-terminal end of a high heel primer represented by SEQ ID NO: 1, was mixed with less than 1 μg of total RNA, and the mixture was allowed to react at 65° C. for 10 minutes so as to anneal the poly dT-high heel primer in the total RNA. Then, the annealed RNA was added with enzyme reactant including transcriptase (400 unit, Finzyme, Finland), dNTP mixture (10 mM, Solgent, Korea), RNAsin (5 unit, Promega, USA), and the like, and subjected to reverse transcription reaction at 42° C. for 2 hours, so as to synthesize cDNA. In this step, an RNA/cDNA hybrid is formed.

The primer of SEQ ID NO: 2 is the poly dT-high heel primer used in this Example. In this primer, V is one base selected from the group consisting of A, G and C, and N is one base selected from the group consisting of A, T, G and C.

SEQ ID NO: 1: 5′-CGC TGG GCC GAC CGG GCG CGG
GAC-3′
SEQ ID NO: 2: 5′-CTA CGC TGG GCC GAC CGG GCG CGG
GAC TTT TTT TTT TTT TTT TTT TTV N-3′

### Example 2

### Synthesis of Double-Stranded cDNA

To the RNA/cDNA hybrid synthesized in Example 1, enzyme reactant including RNase H (2 unit, Invitrogen, USA), DNA polymerase (40 unit, Invitrogen, USA), DNA ligase (6 unit, Invitrogen, USA), dNTP mixture (10 mM, Invitrogen, USA) and the like, was added, and the mixture was allowed to react at 16° C. for 2 hours, so as to synthesize a double-stranded cDNA. Then, the synthesized cDNA was treated with 7.5 μl of 1M NaOH/2 mM EDTA at 65° C. for 10 minutes.

In order to extract only synthesized double-stranded cDNA, 100 μl of a solution of the synthesized sample in nuclease-free water was mixed with 200 μl of PCI (25:24:1, Sigma, USA) in a phase-lock gel tube (Eppendorf, Germany), and the mixture was centrifuged at 13,000 rpm for 5 minutes. The aqueous phase was transferred into a fresh tube, and precipitated at −70° C. for 5 minutes by the addition of 0.5 parts of 7.5M ammonium acetate, 1 μl of linear acrylamide and 2.5 parts of EtOH, followed by centrifugation at 13,000 rpm for 15 minutes. The resulting material was washed with 500 μl of EtOH, and EtOH was removed, after which the remaining material was dried. Then, the dried material was dissolved in 10 μl of nuclease-free water.

### Example 3

### Linear Amplification

The double stranded cDNA extracted in Example 2 was added with 2 μg of a high-heel primer of SEQ ID NO: 1 (Bioneer, Korea) and enzyme reactant including Taq polymerase (5 unit, Solgent, Korea), dNTP (10 mM dATP, 10 mM dCTP, 10 mM dGTP, 2 mM dTTP, and 8 mM aminoally1-dUTP) and the like and subjected to linear PCR amplification. The PCR amplification consisted of denaturation at 94° C. for 10 minutes, followed by 35 cycles of 45 seconds at 94° C. and 2 minutes at 72° C., and then extension at 72° C. for 5 minutes. The linear PCR amplification product was isolated and purified with a PCR purification kit (Intron, Inc., Korea).

As defined herein, the high heel primer used above is a primer which is designed to be annealed at a temperature of 65-75° C. and preferably 72° C. Also, it can optimize a linear amplification process since the efficiency of Taq polymerase is maximized at 72° C. Also, it can eliminate the risk of exponential amplification since the high heel primer is annealed at high temperature. In addition, it can increase the time efficiency of the process, since the annealing of the high-heel primer and the extension of cDNA to be synthesized can occur at the same time.

### Example 4

### Application to Microarray

The cDNA sample which had been linearly amplified in Example 3 was concentrated with Microcon-30 (Millipore, USA) until its volume reaches 10 μl. Then, the concentrated sample was mixed with fluorescent material (Cy-3 and/or Cy-5), and reacted with 0.5 μl of 1M NaHCO₃ (pH 9.0) in a dark place for 1 hour. The reaction mixture was quenched with 4.5 μl of 4M hydroxylamine for 15 minutes in a dark place. The fluorescence-labeled cDNA was isolated and purified again with a PCR purification kit (Intron, Inc., Korea).

The isolated and purified cDNA was added with 20 μl of human cot-1 (2 ∥g/∥l, Invitrogen, USA), 2 μl of poly A (20 μg/μl, Sigma, USA) and 2 μl of yeast tRNA (20 μg/μl, Invitrogen, USA), and then concentrated with Microcon-30 (Millipore, Inc., USA) until it reaches a suitable volume. The concentrated sample was transferred into a fresh tube, and titrated to the final hybridization volume, and then mixed with 5×SSC, 0.1% SDS, and 30-50% formamide. The resulting sample was denatured at 100° C. for 2 minutes, and then subjected to a microarray test.

The microarray test may also be performed by inserting a monofunctional fluorescent material during the linear amplification process. In particular, the obtained double-stranded cDNA may also be applied to a microarray of various platforms by inserting Cy-3-dUTP/Cy-5-dUTP into the sites between which cDNA is synthesized, so as to label the cDNA with each fluorescent color.

### Example 5

### Linear Amplification of Small Amounts of RNA

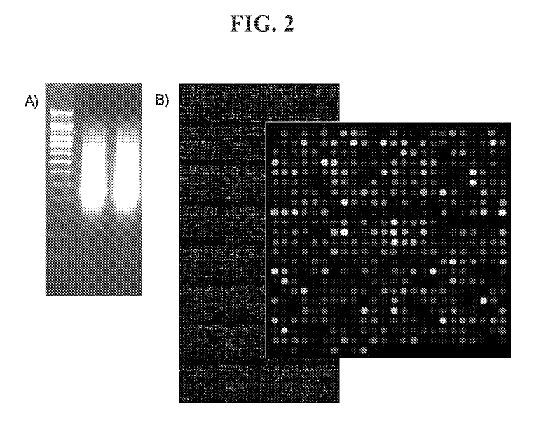

Each 1 μg of total RNA of a control group [293 (ATCC CRL1573)] and a test group [HeLa (ATCC CCL2)] was subjected to linear amplification by the inventive method (FIG. 2). FIG. 2(A) shows the results of 1% agarose gel electrophoresis for 0.1 part of the total amount of the final products. As shown in FIG. 2(A), a main product had about a 500 bp size, and other products had various sizes ranging from about 300 bp to about 1.3 kb. FIG. 2(B) shows the results of 17K cDNA microarray (GenomicTree, Inc., Korea) tests for final products which have been purified and coupled with a fluorescent dye. Specifically, FIG. 2(B) shows images obtained by coupling the control group sample and the test group sample with a green color dye (Cy-3, Amersham, England) and a red color dye (Cy-5, Amersham, England), respectively, and applying the labeled samples on a human 17K microarray, and then scanning the applied samples. As shown in FIG. 2(B), almost all genes on the microarray were covered.

From such results, it could be found that almost all transcripts present in the sample are uniformly amplified. This suggests that transcripts with the minimum copy number cannot be confirmed for their expression by the prior method, but can be efficiently amplified and detected by the inventive linear amplification method.

### Example 6

### Reproducibility

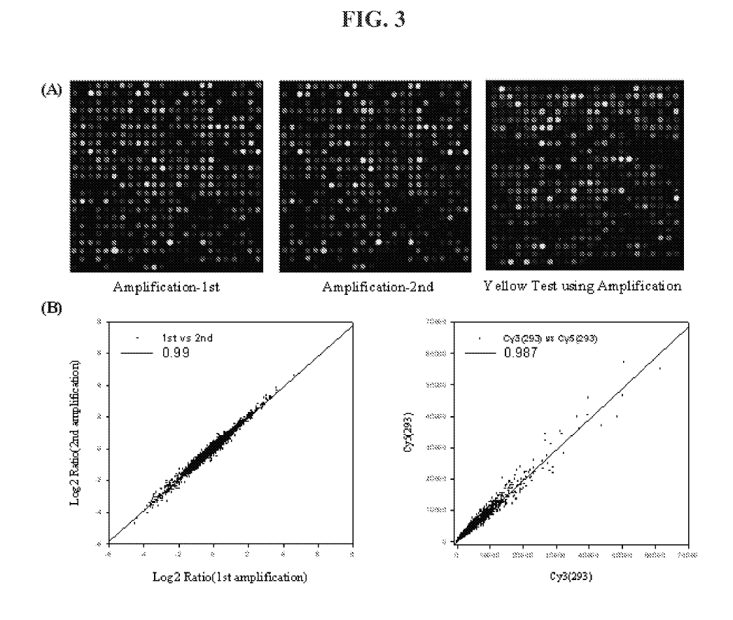

A small amount (1 μg) of total RNA was amplified by the inventive linear amplification method, and the inventive method was confirmed for its reproducibility (FIG. 3). The test was independently conducted two times using RNA of the same origin, and the yellow test was independently conducted only using 293 (ATCC CRL1573) RNA.

FIG. 3(A) shows not only the results of a test where target which had been subjected to two independent linear amplifications (1st & 2nd) was applied on a 17K human cDNA microarray (GenomicTree, Inc., Korea), but also the results of a yellow test where a control group 293 (ATCC CRL1573) RNA alone was applied on the same microarray. As shown in FIG. 3(A), it could be found that two independent test images were very much in agreement with each other, and the results of the yellow test showed that RNA was amplified in almost the same manner regardless of a difference in the dyes (Cy-3 and Cy-5). It is already known that the difference of dyes results in a variety in labeling efficiency, and there were several attempts to overcome errors resulting from this variety.

In FIG. 3(B), the left graph numerically shows the results of FIG. 3(A). 6928 genes showing a signal intensity of more than a given value in the two tests (1st and 2nd amplifications) were selected. The correlation between the two tests which had been independently conducted was examined using the selected genes, and the results showed a high reproducibility of about 0.99. These results indicate that the inventive method has a very excellent reproducibility as compared to the other prior method. Furthermore, the right graph of FIG. 3(B) shows the correlation between the results of a yellow test using the 293 (ATCC CRL1573) RNA sample coupled with Cy3 and using the 293 (ATCC CRL1573) RNA sample coupled with Cy5 dyes. The yellow test results showed that the Cy3[293(ATCC CRL1573) sample] and the Cy5[293(ATCC CRL1573) sample], which used different dyes in relation to one another have 0.987 of correlation, indicating that the inventive method has little or no variability with a change in dyes.

As a result, it could be found that the inventive linear amplification method not only shows excellent reproducibility but also minimizes variety, such as a difference in dyes, the efficiency of enzyme and the like, which can occur in tests.

### Example 7

### Linearity

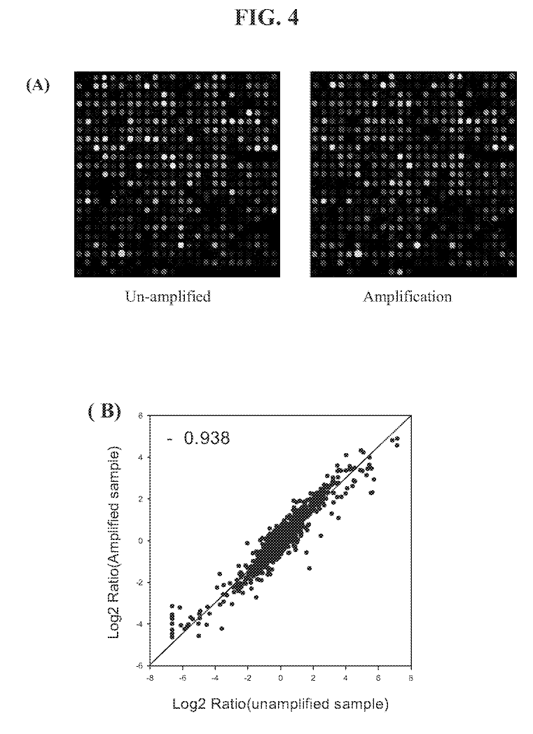

In order to verify the linearity of the inventive linear amplification method, control group [293(ATCC CRL1573)] and test group [HeLa (ATCC CCL2)] samples which had been labeled by different methods were hybridized and the results were analyzed (FIG. 4). As already described above, one of the most important considerations in a sample preparation process for the study of expression patterns of a large amount of genes is that the relative representation of all transcripts present in the sample must be efficiently and reproducibly performed regardless of the total amount of RNA used. For this reason, the linearity of amplification methods is very important.

In FIG. 4(A), the left image shows an array where 100 μg of total RNA have been hybridized with directly labeled cDNA, and the right image shows the same section of an array where 1 μg of total RNA have been subjected to linear amplification by the method of the present invention and then hybridized with cDNA. FIG. 4(B) is a graphic diagram which numerically shows the comparison of correlation between the results of the test using 100 μg of total RNA and the results of the test using 1 μg of linearly amplified RNA. The linearity in FIG. 4(B) was 0.938.

As can be seen in (A) and (B) of FIG. 4, the results of the case where 100 μg of directly labeled total RNA have been hybridized were highly consistent with the results of the case where 1 μg of total RNA was linearly amplified and then hybridized.

Furthermore, as shown in Table 1, the results of a test using 100 μg of total RNA were compared to the results of a test using 1 μg of linearly amplified RNA. 2542 genes meaningful in each of the tests were selected and the value of the correlation between the two tests was examined using the selected genes. The results provide evidence that the linear amplification method according to the present invention has high linearity. This indicates that the inventive linear amplification method provides the relative representation of a difference in the expression of transcripts between each of different samples in a very exact manner.

TABLE 1

| | Direct vs. | Direct vs. |
|---|---|---|
| | amplification (1st) | amplification (2nd) |
| Correlation coefficient | 0.938 | 0.94 |
| Number of significant | 2,542 | 2,542 |
| transcripts | | |

### Example 8

### Changes in Expression Pattern and Signal Intensity According to the Amount of RNA

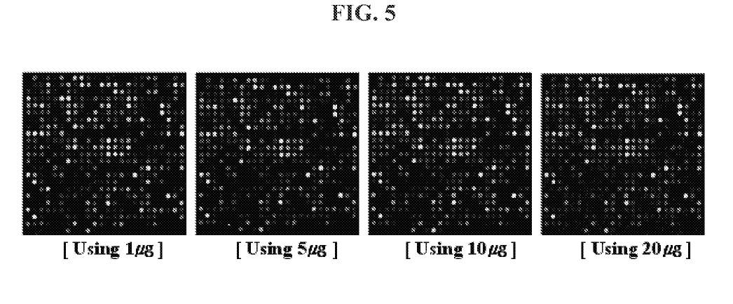

In order to examine if expression pattern and signal intensity are changed depending on the amount of RNA, varied amounts (1 μg, 5 μg, 10 μg and 20 μg) of control group [293 (ATCC CRL1573)] and test group [HeLa (ATCC CCL2)] sample RNAs were linearly amplified by the inventive method, hybridized to a 17K human cDNA microarray (GenomicTree, Inc., Korea), and then examined for their images (FIG. 5). As depicted in FIG. 5, the results showed that signal intensity was maintained constant regardless of the amount of initial sample RNA used in the test, and additionally the expression pattern was maintained constant.

Moreover, as shown in Table 2, 3,153 genes meaningful in each test were selected and the correlation between the tests using 5 μg, 10 μg and 20 μg of RNA, respectively, was examined based on a case where 1 μg of RNA was used. The results showed a high correlation between the tests. These results provide evidence that the inventive linear amplification method shows a high correlation between RNAs of the same origin regardless of the amount and purification method of RNA.

RNA is generally very unstable unlike DNA, and thus, a possibility of nonspecific damages to sample RNA as a result of sample purification, labeling processing or storage processing is very high. For this reason, it cannot be assured that the amount of RNA of control group and comparative group samples at the initial stage of tests exactly agrees, and it is impossible to conduct measurements. Furthermore, in clinical samples processed by methods such as microdisection, there are many cases in which it is difficult to even measure the amount of RNA in order to start a test. Accordingly, it is very important to minimize the effect of such factors that affect the variability of test results. As shown in FIG. 5 and Table 2, the inventive method shows excellent results and can minimize variability caused by other factors than samples.

TABLE 2

| | 1 μg vs. | 1 μg vs. | 1 μg vs. |
|---|---|---|---|
| | 5 μg | 10 μg | 20 μg |
| Correlation coefficient | 0.963 | 0.969 | 0.945 |
| Number of significant transcripts | 3,153 | 3,153 | 3,153 |

### Example 9

### Sensitivity

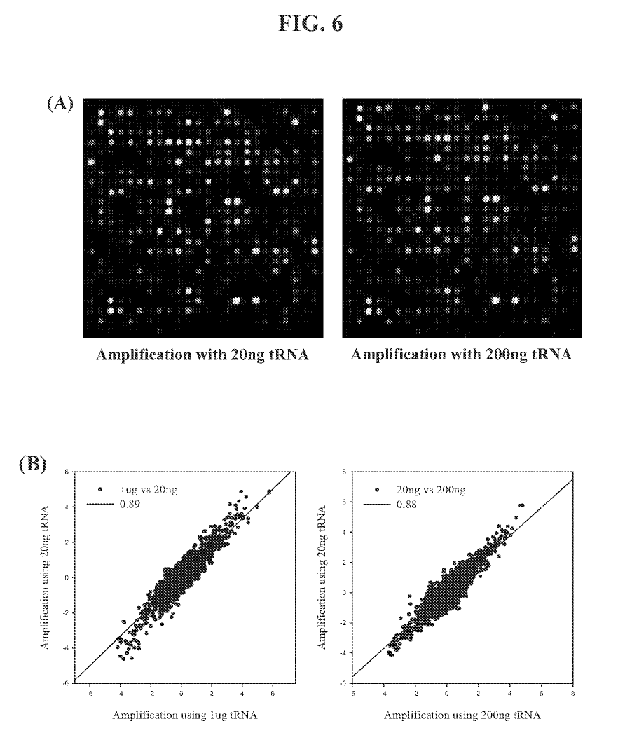

In order to examine the sensitivity of the inventive linear amplification method, each amount of sample RNAs of control group [293 (ATCC CRL1573)] and test group [HeLa (ATCC CCL2)] was diluted, and each of 200 ng and 20 ng of the samples was linearly amplified. The amplified targets then were hybridized to a 17K human cDNA microarray (GenomicTree, Inc., Korea) and examined for their images (FIG. 6).

As shown in FIG. 6(A), the results provide evidence that even 20 ng of RNA can be successfully amplified and detected. The test results using 200 ng RNA and the test results using 20 ng RNA were compared in terms of correlation coefficient, and the comparison result showed a generally high correlation of 0.88 (right graph of FIG. 6(B)). Furthermore, the test results using 20 ng RNA was compared with the test results using 1 μg RNA of the same origin, and the comparison result showed a high correlation of 0.89, indicating that the inventive method has excellent linearity (left graph of FIG. 6(B)).

As described above in detail, the inventive linear amplification method allows a sufficient amount of target for microarray tests to be obtained from a nanogram level of total RNA by one linear amplification process using only one high-heel primer. Also, the inventive method has high sensitivity and can achieve linear amplification in a short time at a lower cost as compared to the prior linear amplification method. Furthermore, since the inventive method uses a DNA primer rather than using an RNA primer, it has an advantage that a linear amplification process is stable. Also, since the inventive method shows a high correlation between RNAs of the same origin regardless of varieties resulting from differences in RNA amounts and dyes, it is very useful in diagnosis with a small amount of sample. Also, since the amplification results of the inventive method are stable, the inventive method is useful in amplifying and detecting a very small amount of RNA from either samples by microdisection or paraffin block samples.

The present invention is very useful in fields which the amplification technology for a small amount of sample is necessarily required. Examples of such fields include DNA microarray tests using a small amount of sample RNA to discover markers of diseases, tests requiring high sensitivity, such as the establishment of gene expression mechanisms, molecular diagnosis using DNA or RNA expression, toxicogenomic studies and pharmacogenomic studies.

While the present invention has been described with reference to particular illustrative embodiments, it is not to be restricted by such embodiments but only by the appended claims. It is to be appreciated that those skilled in the art can change or modify the embodiments herein disclosed without departing from the scope and spirit of the present invention.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method for linear amplification of RNA, the method comprising the steps of:
(a) adding a poly dT-high heel primer, obtained by binding poly dT to the 3′-terminal end of a high-heel primer, to sample RNA, and then allowing the mixture to react at a temperature of about 65-75° C. so as to anneal the poly dT-high heel primer with the poly-A portion of the sample RNA;
(b) reacting the annealed sample with reverse transcriptase reactant so as to synthesize a cDNA, thus forming an RNA/cDNA hybrid;
(c) reacting the formed RNA/cDNA hybrid with enzyme reactant including RNaseH, DNA polymerase and DNA ligase, so as to synthesize a double-stranded cDNA; and
(d) adding a high-heel primer, dNTP and DNA polymerase to the double-stranded cDNA and subjecting the mixture to linear PCR amplification, in which annealing and extension are performed at about the same temperature in a range of 65-75° C.

2. The method for the linear amplification of RNA according to claim 1, wherein the high-heel primer has Tₘ of about 65 to 75° C.

3. The method according to claim 2, wherein the Tm is about 68 to 74° C.

4. The method according to claim 3, wherein the Tm is about 72° C.

5. The method according to claim 1, wherein the CG content is at least about 70%.

6. The method according to claim 5, wherein the CG content is at least about 75%.

7. The method according to claim 1, wherein the high-heel primer is about 10 to about 50 nucleotides long.

8. The method according to claim 7, wherein the high-heel primer is about 10 to about 40 nucleotides long.

9. The method according to claim 1, wherein the annealing and extension in the step (d) is performed at about 65 to 75° C.

10. The method according to claim 1, wherein the annealing and extension in the step (d) is performed at about 68 to 74° C.

11. The method according to claim 6, wherein the annealing and extension in the step (d) is performed at about 72° C.

12. The method for the linear amplification of RNA according to claim 1, wherein the step (d) of amplifying the double-stranded cDNA additionally comprises adding aminoally1-dUTP to the cDNA and labeling the cDNA with a monofunctional fluorescent substance.

13. The method for the linear amplification of RNA according to claim 1, wherein the DNA polymerase in step (d) comprises Taq polymerase.

14. A system for linear amplification of RNA, the system comprising:
(a) a poly dT-high heel primer, wherein the high heel portion of the primer has a Tm of about 65 to 75° C., CG content of at least about 70% and length of about 10 to about 50 nucleotides to anneal to the poly-A portion of the sample RNA;
(b) reverse transcriptase reactant so as to synthesize a cDNA from the annealed RNA, thus forming an RNA/cDNA hybrid;
(c) enzyme reactants including RNaseH, DNA polymerase and DNA ligase, so as to synthesize a double-stranded cDNA; and
(d) a high-heel primer, dNTP and DNA polymerase, in a mixture or in separate containers to be added to the double-stranded cDNA for linear PCR amplification, in which annealing and extension are performed at about the same temperature in a range of 65-75° C.

15. The system according to claim 14, wherein the Tm is about 68 to 74° C.

16. The system according to claim 15, wherein the Tm is about 72° C.

17. The system according to claim 14, wherein the CG content is at least about 75%.

18. The system according to claim 14, wherein the high-heel primer is about 10 to about 40 nucleotides long.

19. The system according to claim 14, wherein part (d) additionally comprises aminoally1-dUTP and a monofunctional fluorescent substance.

20. The system according to claim 19, wherein the DNA polymerase in part (d) comprises Taq polymerase.


SEQUENCE LISTING (2 sequences)

SEQ ID NO: 1 - DNA, 24 bp, Artificial sequence
  Primer
cgctgggccgaccgggcgcgggac

SEQ ID NO: 2 - DNA, 49 bp, Artificial sequence
  Primer
ctacgctgggccgaccgggcgcgggacttttttttttttttttttttvn